(19) United States
(12) Patent Application Publication
(10) Pub. No.: US 2005/0255467 A1
(43) Pub. Date: Nov. 17, 2005

(54) Methods and computer program products for the quality control of nucleic acid assay

(76) Inventors: Peter Adorjan, Berlin (DE); Fabian Model, Berlin (DE); Thomas Konig, Berlin (DE); Christian Piepenbrock, Berlin (DE); Klaus Junemann, Berlin (DE); Matthias Burger, Berlin (DE); Susanne Schwenke, Berlin (DE)

Correspondence Address: KRIEGSMAN & KRIEGSMAN, 665 FRANKLIN STREET, FRAMINGHAM, MA, 01702, US

(21) Appl. No.: 10/509,449

(22) PCT Filed: Mar. 28, 2003
(86) PCT No.: PCT/EP03/03288

§ 371 Date: Jun. 2, 2005

Related U.S. Application Data
(60) Provisional application No. 60/368,452, filed on Mar. 28, 2002.

(51) Int. Cl. C12Q 1/68

(52) U.S. Cl. 435/6

(57) ABSTRACT

The disclosed invention provides methods and computer program products for the improved verification and controlling of assays for the analysis of nucleic acid variations by means of statistical process control. The invention is characterised in that variables of each experiment are monitored by measuring deviations of said variables from a reference data set and wherein said experiments or batches thereof are indicated as unsuitable for further interpretation if they exceed predetermined limits.

### TECHNICAL FIELD

The field of the invention relates to methods and computer program products for the control of assays for the analysis of nucleic acid within DNA samples.

### BACKGROUND ART

A fundamental goal of genomic research is the application of basic research into the sequence and functioning of the genome to improve healthcare and disease management. The application of novel disease or disease treatment markers to clinical and/or diagnostic settings often requires the adaptation of suitable research techniques to large scale high throughput formats. Such techniques include the use of large scale sequencing, mRNA analysis and in particular nucleic acid microarrays. DNA microarrays are one of the most popular technologies in molecular biology today. They are routinely used for the parallel observation of the mRNA expression of thousands of genes and have enabled the development of novel means of marker identification, tissue classification, and discovery of new tissue subtypes. Recently it has been shown that microarrays can also be used to detect DNA methylation and that results are comparable to mRNA expression analysis, see for example P. Adoian et al. Tumour class prediction and discovery by microarray-based DNA methylation analysis. Nucleic Acid Research, 30(5), 02. and T. Golub et al. Molecular classification of cancer: Class discovery and class prediction by gene expression monitoring. Science, 286:531-537, 1999.

Despite the popularity of microarray technology, there remain serious problems regarding measurement accuracy and reproducibility. Considerable effort has been put into the understanding and correction of effects such as background noise, signal noise on a slide and different dye efficiencies see for example C. S. Brown et al. Image metrics in the statististical analysis of dna microarray data Proc Natl Acad Sci USA, 98(16):8944-8949, July 2001 and G. C. Tseng et al. Issues in cdna microarray analysis: Quality filtering, channel normalization, models of variations and assessment of gene effects. Nucleic Acids Research, 29(12):2549-2557, 2001. However, with the exception of overall intensity normalization (A. Zien et al. Centralization: A new method for the normalization of gene expression data. Proc. ISMB '01/Bioinformatics, 17(6):323-331, 2001), it is not clear how to handle variations between single slides and systematic alterations between slide batches. Between slide variations are particularly problematic because it is difficult to explicitly model the numerous different process factors which may distort the measurements. Some examples are concentration and amount of spotted probe during array fabrication, the amount of labeled target added to the slide and the general conditions during hybridization. Other common but often neglected problems are handling errors such as accidental exchange of different probes during array fabrication. These effects can randomly affect single slides or whole slide batches. The latter is especially dangerous because it introduces a systematic error and can lead to false biological conclusions.

There are several ways to reduce between slide variance and systematic errors. Removing obvious outlier chips based on visual inspection is an easy and effective way to increase experimental robustness. A more costly alternative is to do repeated chip experiments for every single biological sample and obtain a robust estimate for the average signal. With or without chip repetitions randomized block design can further increase certainty of biological findings. Unfortunately, there are several problems with this approach. Outliers can not always be detected visually and it is not feasible to make enough chip repetitions to obtain a fully randomized block design for all potentially important process parameters. However, when experiments are standardized enough, process dependent alterations are relatively rare events. Therefore instead of reducing these effects by repetitions one should rather detect problematic slides or slide batches and repeat only those. This can only be achieved by controlling process stability.

Maintaining and controlling data quality is a key problem in high throughput analysis systems. The data quality is often hampered by experiment to experiment variability introduced by the environmental conditions that may be difficult to control.

Examples of such variables include, variability in sample preparation and uncontrollable reaction conditions. For example, in the case of micro array analysis systematic changes in experimental conditions across multiple chips can seriously affect quality and even lead to false biological conclusions. Traditionally the influence of these effects has been minimized by expensive repeated measurements, because a detailed understanding of all process relevant parameters appears to be an unreasonable burden.

Process stability control is well known in many areas of industrial production where multivariate statistical process control (MVSPC) is used routinely to detect significant deviations from normal working conditions. The major tool of MVSPC is the T² control chart, which is a multivariate generalization of the popular univariate Shewhart control procedure.

See for example U.S. Pat. No. 5,693,440. In this application Hotelling's T2 in combination with a simple PCA was used as a means of process verification in photographic processes. Although this application demonstrates the use of simple principle component analysis, the benefits of this are not obvious as the data set was not of a high dimensionality as is often encountered in biotechnological assays such as sequencing and microarray analysis. Furthermore, this application recommends the application of PCA on the “cleared” reference data set, which may hide variations caused by the data set to be monitored.

The application of MVSPC for statistical quality control of microarray and high throughput sequencing experiments is not straightforward. This is because most of the relevant process parameters of a microarray experiment cannot be measured routinely in a high throughput environment.

5-methylcytosine is the most frequent covalent base modification of the DNA of eukaryotic cells. Cytosine methylation only occurs in the context of CpG dinucleotides. It plays a role, for example, in the regulation of the transcription, in genetic imprinting, and in tumorigenesis. Methylation is a particularly relevant layer of genomic information because it plays an important role in expression regulation (K. D. Robertson et al. DNA methylation in health and disease. Nature Reviews Genetics, 1:11-19, 2000). Methylation analysis has therefore the same potential applications as mRNA expression analysis or proteomics. In particular DNA methylation appears to play a key role in imprinting associated disease and cancer (see for example, Zeschnigk M, Schmitz B, Dittrich B, Buiting K, Horsthemke B, Doerfler W. “Imprinted segments in the human genome: different DNA methylation patterns in the Prader-Willi/Angelman syndrome region as determined by the genomic sequencing method” Hum Mol Genet 1997 March; 6(3):387-95 and Peter A. Jones “Cancer. Death and methylation”. Nature. 2001 Jan. 11; 409(6817):141, 143-4. The link between cytosine methylation and cancer has already been established and it appears that cytosine methylation has the potential to be a significant and useful clinical diagnostic marker.

The application of molecular biological techniques in the field of methylation analysis have hereto been limited to research applications, to date it is not a commercially utilised clinical marker. The application of methylation disease markers to a large scale analysis format suitable for clinical, diagnostic and research purposes requires the implementation and adaptation of high throughput techniques in the field of molecular biology to the specific constraints and demands specific to methylation analysis. Preferred techniques for such analyses include the analysis of bisulfite treated sample DNA by means of micro array technologies, and real time PCR based methods such as MethyLight and HeavyMethyl.

### DISCLOSURE OF INVENTION

### BRIEF DESCRIPTION

The described invention provides a novel method and computer program products for the process control of assays for the analysis of nucleic acid within DNA samples. The method enables the estimation of the quality of an individual assay based on the distribution of the measurements of variables associated with said assay in comparison to a reference data set. As these measurements are extremely high dimensional and contain outliers the application of standard MVSPC methods is prohibited. In a particularly preferred embodiment of the method a robust version of principle component analysis is used to detect outliers and reduce data dimensionality. This step enables the improved application of multivariate statistical process control techniques. In a particularly preferred embodiment of the method, the T² control chart is utilised to monitor process relevant parameters. This can be used to improve the assay process itself, limits necessary repetitions to affected samples only and thereby maintains quality in a cost effective way.

### DETAILED DESCRIPTION

In the following application the term ‘statistical distance’ is taken to mean a distance between datasets or a single measurement vector and a data set that is calculated with respect to the statistical distribution of one or both data sets. In the following the term ‘robust’ when used to describe a statistic or statistical method is taken to mean a statistic or statistical method that retains its usefulness even when one or more of its assumptions (e.g. normality, lack of gross errors) is violated.

The method and computer program products according to the disclosed invention provide novel means for the verification and controlling of biological assays. Said method and computer program products may be applied to any means of detecting nucleic acid variations wherein a large number of variables are analysed, and/or for controlling experiments wherein a large number of variables influence the quality of the experimental data Said method is therefore applicable to a large number of commonly used assays for the analysis of nucleic acid variations including, but not limited to, microarray analysis and sequencing for example in the fields of mRNA expression analysis, single nucleotide polymorphism detection and epigenetic analysis.

To date, the automated analysis of nucleic acid variations has been limited by experiment to experiment variation. Errors or fluctuations in process variables of the environment within which the assays are carried out can lead to decreased quality of assays which may ultimately lead to false interpretations of the experimental results. Furthermore, cerin constraints of assay design, most notably nucleic acid sequence (which affects factors such as cross hybridisation, background and noise in microarray analysis), may be subject to experiment to experiment variation further complicating standard means of assay result analysis and data interpretation.

One of the factors that complicates the controlling of such high throughput assays within predetermined parameters is the high dimensionality of the datasets which are required to be monitored. Therefore, multiple repetitions of each assay are often carried out in order to minimize the effects of process artefacts in the interpretation of complex nucleic acid assays. There is therefore a pronounced need in the art for improved methods of insuring the quality of high throughput genomic assays.

In one embodiment, the method and computer program products according to the invention provide a means for the improved detection of assay results which are unsuitable for data interpretation. The disclosed method provides a means of identifying said unsuitable experiments, or batches of experiments, said identified experiments thereupon being excluded from subsequent data analysis. In an alternative embodiment said identified experiments may be further analysed to identify specific operating parameters of the process used to carry out the assay. Said parameters may then be monitored to bring the quality of subsequent experiments within predetermined quality limits. The method and computer program products according to the invention thereby decrease the requirement for repetition of assays as a standard means of quality control. The method according to the invention further provides a means of increasing the accuracy of data interpretation by identifying experiments unsuitable for data analysis.

In the following it is particularly preferred that all herein described elements of the method are implemented by means of a computer.

The aim of the invention is achieved by means of a method of verifying and controlling nucleic acid analysis assays using statistical process control and/or and computer program products used for said purpose. The statistical process control may be either multivariate statistical process control or univariate statistical process control. The suitability of each method will be apparent to one skilled in the art. The method according to the invention is characterized in that variables of each experiment are monitored, for each experiment the statistical distance of said variables from a reference data set (also herein referred to as a historical data set) are calculated and wherein a deviation is beyond a pre-determined limit said experiment is indicated as unsuitable for further interpretation. It is particularly preferred that the method according to the invention is implemented by means of a computer.

In a preferred embodiment this method is used for the controlling and verification of assays used for the determination of cytosine methylation patterns within nucleic acids. In a particularly preferred embodiment the method is applied to those assays suitable for a high throughput format, for example but not limited to, sequencing and microarray analysis of bisulphite treated nucleic acids.

In one embodiment, the method according to the invention comprises four steps. In the first step a reference data set (also herein referred to as a historical data set) is defined, said data set consisting of all the variables that are to be monitored and controlled. In the second step a test data set is defined. Said test data set consists of the experiment or experiments that are to be controlled, and wherein each experiment is defined according to the values of the variables to be analysed.

In the third step of the method the statistical distance between the reference and test data sets or elements or subsets thereof are determined. In the fourth step of the method individual elements or subsets of the test dataset which have a statistical distance larger than that of a predetermined value are identified.

In a particularly preferred embodiment of the method, subsequent to the definition of the reference and test data sets the method comprises a further step, hereinafter referred to as step 2ii). Said step comprises reducing the data dimensionality of the reference and test data set by means of robust embedding of the values into a lower dimensional representation. The embedding space may be calculated by using one or both of the reference and the test data set. It is particularly preferred that the data dimensionality reduction is carried out by means of principle component analysis. In one embodiment of the method step bii) comprises the following steps. In the first step the data set is projected by means of robust principle component analysis. In the second step outliers are removed from the data set according to their statistical distances calculated by means of one or more methods taken from the group consisting of: Hotelling's T² distance; percentiles of the empirical distribution of the reference data set; Percentiles of a kernel density estimate of the distribution of the reference data set and distance from the hyperplane of a nu-SVM (see Schlkopf, Bernhard and Smola, Alex J. and Williamson, Robert C. and Bartlett, Peter L., New Support Vector Algorithms. Neural Computation, Vol. 12, 2000.), estimating the support of the distribution of the reference data set. In the third step the embedding projection is calculated by means of standard principle component analysis and the cleared or the complete data set is projected onto this basis vector system.

In one embodiment of the method at least one of the variables measured in steps a) and b) is determined according to the methylation state of the nucleic acids.

In a further preferred embodiment of the method at least one of the variables measured in the first and second steps is determined by the environment used to conduct the assay, wherein the assay is a microarray analysis it is further preferred that these variables are independent of the arrangement of the oligonucleotides on the array. In a particularly preferred embodiment said variables are selected from the group comprising mean background/baseline values; scatter of the background/baseline values; scatter of the foreground values, geometrical properties of the array, percentiles of background values of each spot and positive and negative assay control measures.

In a further preferred embodiment of the method at least one of the variables measured in the first and second steps is determined by the environment used to conduct the assay, wherein the assay is a microarray analysis it is further preferred that these variables are independent of the arrangement of the oligonucleotides on the array.

In a particularly preferred embodiment wherein the assay is a microarray based assay said variables are selected from the group comprising mean background/baseline intensity values; scatter of the background/baseline intensity values; coefficient of variation for background spot intensities, statistical characterisation of the distribution of the background/baseline intensity values (1%, 5%, 10%, 25% 50%, 75% 90%, 95%, 99% percentiles, skewness, kurtosis), scatter of the foreground intensity values; coefficient of variation for foreground spot intensities; statistical characterisation of the distribution of the foreground intensity values (1%, 5%, 10%, 25% 50%, 75% 90%, 95%, 99% percentiles, skewness, kurtosis), saturation of the foreground intensity values, ratio of mean to median foreground intensity values, geometrical properties of the array as in the gradient of background intensity values calculated across a set of consecutive rows or columns along a given direction, mean spot diameter values, scatter of spot diameter values, percentiles of spot diameter value distribution across the microarray, and positive and negative assay control measures.

When selecting appropriate variables for the analysis an important criterion is that the statistical distribution of these variables does not change significantly between different series of experiments (wherein each series of experiments is defined as a large series of measurements carried out within one time period and with the same assay design). This allows the utillisation of measurements from previous studies as reference data sets.

Wherein the assay is a microarray based assay it is preferred that the variables to be analysed include at least one variable that refers to each of the foreground, background, geometrical properties and saturation of the microarray. A particularly preferred set of variables is as follows:
Background
   1. 75% quantile of all observed values of the percentage of background pixel per spot above the mean signal+one standard deviation
   2. 75% quantile of all observed values of the percentage of background pixel per spot above the mean signal+two standard deviations
   3. skewness of the distribution of observed values of the median background intensity per spot
   4. mean value of the ratio of observed values: mean background intensity divided by median background intensity per spot
Geometry
   1. 75% quantile of all observed values of the difference of background intensities of four consequtive rows avereraged and the following 4 consequtive rows
   2. same as in 1. for columns
Spot Characteristic
   1. 95% quantile of all observed spot diameters
   2. median (50% quantile) of all observed spot diameters
   3. 75% quantile of the ratio of observed values defined by: standard deviation of foreground intensity per spot divided by mean of foreground intensity per spot
   4. median of the ratio of all observed values defined by: mean foreground intensity per spot divided by median foreground intensity per spot
Saturation
   1. 95% quantile of foreground intensity pixel saturation percentage per spot values

For each variable or group thereof the further steps of the method are according to the described method. Therefore, in one embodiment of the method first calculate the statistical distance of each variable from the reference dataset. It is preferred that the reference data set is composed of a large set of previous measurements, that is obtained under similar experimental conditions. Then combine variables within each category either by embedding into a 1-dimensional space or by averaging single values.

Preferably, both the statistical distance and the embedding is carried out in a robust way.

In a further preferred embodiment the to calculate quality of the experiment first calculate a lower dimensional embedding of both the reference and the test data seL It is preferred that the reference data set that is used is composed of a large set of previous measurements, that are obtained under similar experimental conditions. Secondly, calculate the statistical distance in this reduced dimensional space. Use this statistical distance as the quality score.

It will be obvious to one skilled in the art that is not necessary that the second step of the method is temporally subsequent to the first step of the method. The reference data set may be defined subsequent to the test data set, alternatively it may be a defined concurrently with the test data set. In one embodiment of the method the reference data set may consist of all experiments run in a series wherein said series is user defined. To give one example, where a microarray assay is applied to a series of tissue samples the measured variables of all the samples may be included in said reference data set, however analyses of the same tissue set using an alternative array may not. Accordingly the test data set may be a subset of or identical to the reference data set. In another embodiment of the method the reference data set consists of experiments that were carried out independent or separate from those of the test data set. The two data sets may be differentiated by factors such as but not limited to time of production, operator (human or machine), environment used to carry out the experiment (for example, but not limited to temperature, reagents used and concentrations thereof, temporal factors and nucleic acid sequence variations).

In a further embodiment of the method the reference data set is derived from a set of experiments wherein the value of each analysed variable of each experiment is either within predetermined limits or, alternatively, said variables are controlled in an optimal manner.

In step 4 of the method the statistical distance may calculated by means of one or more methods taken from the group consisting of the Hotelling's T² distance between a single test measurement vector and the reference data set, the Hotelling'-T² distance between a subset of the test data set and the reference data set, the distance between the covariance matrices of a subset of the test data set and the covariance matrix of the reference set, percentiles of the empirical distribution of the reference data set and percentiles of a kernel density estimate of the distribution of the reference data set, distance from the hyperplane of a nu-SVM (see Schlkopt, Bernhard and Smola, Alex J. and Williamson, Robert C. and Bartlett, Peter L., New Support Vector Algorithms. Neural Computation, Vol. 12, 2000.), estimating the support of the distribution of the reference data set. Wherein Hotelling's T² distance between a single test measurement vector and the reference data set is measured, it is preferred that the T² distance is calculated by using the sample estimate for mean and variance or any robust estimate for location, including trimmed mean, median, Tukey's biwight, 11-median, Oja-median, minimum volume ellipsoid estimator and Sestimator (see Hendrik P. Lopuhaa and Peter J. Rousseeuw: Breakdown points of affine equivariant estimators of multivariate location and covariance matrices) and any robust estimate for scale including Median Absolute Deviation, interquantile range Qn-estimator, minimum volume ellipsoid estimator and S-timator.

In a particularly preferred embodiment this is defined as:
T²(i)=(mᵢ−μ)′S⁻¹(mᵢ−μ)
wherein reference set mean

$$μ = ( 1 / {N}_{C} ) {\sum }_{i = 1}^{{N}_{C}} {m}_{i}$$

and the reference set sample covariance matrix

$$S = 1 / ( {N}_{C} - 1 ) {\sum }_{i = 1}^{{N}_{C}} ( {m}_{i} - μ ) {( {m}_{i} - μ )}^{′}$$

wherein N_{c} is the number of experiments in the reference set and mᵢ is the is the ith measurement vector of the reference or test data set.

Wherein the Hotelling'-T² distance is calculated between a subset of the test data set and the reference data set, it is preferred that the T² is calculated by using the sample estimate for mean and variance or any robust estimate for location, including trimmed mean, median, Tukey's biwight, 11-median, Oja-median and any robust estimate for scale including Median Absolute Deviation, interquantile range Qn-estimator, minimum volume ellipsoid estimator and S-estimator. In a particularly preferred embodiment this is defined as:
T_{w}²(i)=(μ_{HDS}−μ_{CDS})^{T}\overline{S}⁻¹(μ_{HDS}−μ_{CDS})
Wherein ‘HDS’ refers to the historical data set, also referred to herein as the reference data set and ‘CDS’ refers to the current data set also referred to herein as the test data set Furthermore, \overline{S} is calculated from the sample covariance matrices S_{HDS} and S_{CDS}

$$\overline{S} = \frac{( {N}_{HDS} - 1 ) {S}_{HDS} + ( {N}_{CDS} - 1 ) {S}_{CDS}}{{N}_{HDS} + {N}_{CDS} - 2} .$$

Wherein the statistical distance is calculated as the distance between the covariance matrices of a subset of the test data set and the covariance matrix of the reference set, it is preferred that the test statistics of the likelihood ratio test for different covariance matrixes are included. See for example Hartung J. and Epelt B: Multivariate Statistik. R. Oldenburg, Miinchen, Wien, 1995. In a particularly preferred embodiment this is defined as:

$$L ( i ) = 2 [ ln \vert \overline{S} \vert - \frac{{N}_{HDS} - 1}{{N}_{HDS} + {N}_{CDS} - 2} ln \vert {S}_{HDS} \vert - \frac{{N}_{CDS} - 1}{{N}_{HDS} + {N}_{CDS} - 2} ln \vert {S}_{CDS} \vert ]$$

In a further embodiment of the method, subsequent to steps 1 to 4, the method may further comprise a fifth step. In a first embodiment of the method said identified experiments or batches thereof are further interrogated to identify specific operating parameters of the process used to carry out the assay that may be required to be monitored to bring the quality of the assays within predetermined quality limits. In one embodiment of the method this is enabled by means of verifying the influence of each individual variable by computing its' univariate T² distances between reference and test data set. In a further embodiment one may analyse the orthogonalized T² distance computing the PCA embedding of step 2ii) based on the reference data set. The principle component responsible for the largest part of the T² distance of an out of control test data point may then be identified. Responsible individual variables can be identified by their weights in this principle component. In a further embodiment variables responsible for the out of control situation can be identified by backward selection. A subset of variables or single variables can be excluded from the statistical distance calculation and one can observe whether the computed distance gets significantly smaller. Wherein the computed statistical distance significantly decreases one can conclude that the excluded variables were at least partially responsible for the observed out of control situation.

In a further embodiment, said identified assays are designated as unsuitable for data interpretation, the experiment(s) are excluded from data interpretation, and are preferably repeated until identified as having a statistical distance within the predetermined limit.

In a particularly preferred embodiment, the method further comprises the generation of a document comprising said elements or subsets of the test data determined to be outliers. In a further embodiment said document further comprises the contribution of individual variables to the determined statistical distance. It is preferred that said document be generated in a readable manner, either to the user of the computer program or by means of a computer, and wherein said computer readable document further comprises a graphical user interface.

Said document may be generated by any means standard in the art, however, it is particularly preferred that the document is automatically generated by computer implemented means, and that the document is accessible on a computer readable format (e.g. HTML, portable document format (pdf), postscript (ps)) and variants thereof. It is further preferred that the document be made available on a server enabling simultaneous access by multiple individuals. In another aspect of the invention computer program products are provided. An exemplary computer program product comprises:
a) a computer code that receives as input a reference data set
b) a computer code that receives as input a test data set
c) a computer code that determines the statistical distance between the reference data set and test data set or elements or subsets thereof
d) a computer code that identifies individual elements or subsets of the test dataset which have a statistical distance larger than that of a predetermined value
e) a computer readable medium that stores the computer code.

It is further preferred that said computer program product comprises a computer code for the reduction of the data dimensionality of the reference and test data set by means of robust embedding of the values into a lower dimensional representation.

In a preferred embodiment the computer program product further comprises a computer code that reduces the data dimensionality of the reference and test data set by means of robust embedding of the values into a lower dimensional representation. In this embodiment of the invention the embedding space may be calculated using one or both of the reference and the test data sets. In one particularly preferred embodiment the computer code carries out the data dimensionality reduction step by means of a method comprising the following steps:
i) Projecting the data set by means of robust principle component analysis
ii) Removing outliers from the data set according to their statistical distances calculated by means of one or more methods taken from the group consisting of: Hotelling's T² distance; percentiles of the empirical distribution of the reference data set; Percentiles of a kernel density estimate of the distribution of the reference data set and distance from the hyperplane of a nu-SVM, estimating the support of the distribution of the reference data set
iii) Calculating the embedding projection by standard principle Component analysis and projecting the cleared or the complete data set onto this basis vector system.

In a further preferred embodiment the computer program product further comprises a computer code that generates a document comprising said elements or subsets of the test data identified by the computer code of step d). It is preferred that said document be generated in a readable manner, either to the user of the computer program or by means of a computer, and wherein said computer readable document further comprises a graphical user interface.

### EXAMPLES

### Example 1

In this example the method according to the invention is used to control the analysis of methylation patterns by means of nucleic acid microarrays.

In order to measure the methylation state of different CpG dinucleotides by hybridization, sample DNA is bisulphite treated to convert all unmethylated cytosines to uracil, this treatment is not effective upon methylated cytosines and they are consequently conserved. Genes are then amplified by PCR using fluorescently labelled primers, in the amplificate nucleic acids unmethylated CpG dinucleotides are represented as TG dinucleotides and methylated CpG sites are conserved as CG dinucleotides. Pairs of PCR primers are multiplexed and designed to hybridise to DNA segments containing no CpG dinucleotides. This allows unbiased amplification of multiple alleles in a single reaction. All PCR products from each individual sample are then mixed and hybridized to glass slides carrying a pair of immobilised oligonucleotides for each CpG position to be analysed. Each of these detection oligonucleotides is designed to hybridize to the bisulphite converted sequence around a specific CpG site which is either originally unmethylated (TG) or methylated (CG). Hybridization conditions are selected to allow the detection of the single nucleotide differences between the TG and CG variants.

In the following, N_{CpG} is the number of measured CpG positions per slide, N_{S} is the number of biological samples in the study and N_{C} is the number of hybridized chips in the study. For a specific CpG position k Î{1, . . . , N_{CpG}}, the frequency of methylated alleles in sample j Î{1, . . . , N_{S}}, hybridized onto chip i Î{1, . . . , N_{C}) can then be quantified as equation 1

$${m}_{ik} = log \frac{{CG}_{ik}}{{TG}_{ik}} ,$$

where CGᵢₖ and TGᵢₖ are the corresponding hybridization intensities. This ratio is invariant to the overall intensity of the particular hybridization experiment and therefore gives a natural normalization of our data.

Here we will refer to a single hybridization experiment i as experiment or chip. The resulting set of measurement values is the methylation profile mᵢ=(mᵢ₁, . . . , m_{iNCpG})′. We usually have several repeated hybridization experiments i for every single sample j. The methylation profile for a sample j is estimated from its set of repetitions Rⱼ by the L₁-median as mⱼ=_{xiÎRj}|mᵢ−x|₂. In contrast to the simple component wise median this gives a robust estimate of the methylation profile that is invariant to orthogonal linear transformations such as PCA.

Data Sets

In our analysis we used data from three microarray studies. In each study the methylation status of about 200 different CpG dinucleotide positions from promoters, intronic and coding sequences of 64 genes was measured.

Temperature Control: Our first set of 207 chips came from a control experiment where PCR amplificates of DNA from the peripheral blood of 15 patients diagnosed with ALL or AML was hybridized at 4 different temperatures (38 C, 42 C, 44 C, 46 C). We will use this data set to prove that our method can reliably detect shifts in experimental conditions.

Lymphoma: The second data set with an overall number of 647 chips came from a study where the methylation status of different subtypes of non-Hodgkin lymphomas from 68 patients was analyzed. All chips underwent a visual quality control, resulting in quality classification as “good” (proper spots and low background), “acceptable” (no obvious defects but uneven spots, high background or weak hybridization signals) and “unacceptable” (obvious defects). We will use this data set to identify different types of outliers and show how our methods detect them.

In addition we simulated an accidental exchange of oligo probes during slide fabrication in order to demonstrate that such an effect can be detected by our method. The exchange was simulated in silico by permuting 12 randomly selected CpG positions on 200 of the chips (corresponding to an accidental rotation of a 24 well oligo supply plate during preparation for spotting).

ALL/AML: Finally we show data from a second study on ALL and AML, containing 468 chips from 74 different patients. During the course of this study 46 oligomeres had to be re-synthesized, some of which showed a significant change in hybridization behavior, due to synthesis quality problems. We will demonstrate how our algorithm successfully detected this systematic change in experimental conditions.

Typical Artefacts

Typical artefacts in microarray based methylation analysis are shown in FIG. 1. The plots show the correlation between single or averaged methylation profiles. Every point corresponds to a single CpG position, the axis-values are log ratios. a) A normal chip, showing good correlation to the sample average. b) A chip classified as “unacceptable” by visual inspection. Many spots showed no signal, resulting in a log ratio of 0. c) A chip classified as “good”. Hybridization conditions were not stringent enough, resulting in saturation. In many cases pairs of CG and TG oligos showed nearly identical high signals, giving a log ratio around 0. d) A chip classified as “acceptable”. Hybridization signals were weak compared to the background intensity, resulting in a high amount of noise. e) Comparison of group averages over all 64 ALL/AML chips hybridized at 42 C and all 48 ALL/AML chips hybridized at 44 C. f) Comparison of group averages over 447 regular chips from the lymphoma data set and the 200 chips with a simulated accidental probe exchange during slide production, affecting 12 CpG positions.

With a high number of replications for each biological sample and the corresponding average m being reliably estimated, outlier chips can be relatively easily detected by their strong deviation from the robust sample average. In the following, we will discuss some typical outlier situations, using data from the Lymphoma experiment. In this case the hybridization of each sample was repeated at a very high redundancy of 9 chips.

After identifying possible error sources the question remains how to reliably detect them, in particular if they can not be avoided with absolute certainty. One aim of the invention is therefore to exclude single outlier chips from the analysis and to detect systematic changes in experimental conditions as early as possible in order to facilitate a fast recalibration of the production process.

Detecting Outlier Chips with Robust PCA

Methods

As a first step we want to detect single outlier chips. In contrast to standard statistical approaches based on image features of single slides we will use the overall distribution of the whole experimental series. This is motivated by the fact that although image analysis algorithms will successfully detect bad hybridization signals, they will usually fail in cases of unspecific hybridization. The aim is to identify the region in measurement space where most of the chips mᵢ, i=1 . . . N_{c}, are located. The region will be defined by its center and an upper limit for the distance between a single chip and the region center. Chips with deviations higher than the upper limit will be regarded as outliers.

A simple approach is to independently define for every CpG position k the deviation from the center μₖ as tₖ=|mᵢₖ−μₖ|sₖ hereinafter referred to as Equation 3, where μₖ=(1/N)ᵢmᵢₖ is the mean and s²ₖ=1/(N−1)ᵢ(mᵢₖ−μₖ)² is the sample variance over all chips. Assuming that the mᵢₖ are normally distributed, tₖ multiplied by a constant follows a t-distribution with N−1 degrees of freedom. This can be used to define the upper limit of the admissible region for a given significance level α.

However, a separate treatment of the different CpG positions is only optimal when their measurement values are independent. As FIG. 2 demonstrates it is important to take into account the correlation between different dimensions. It is possible that a point which is not detected as an outlier by a component wise test is in reality an outlier (e.g. P₁ in FIG. 2). On the other hand, there are points that will be erroneously detected as outliers by a component wise test (e.g. P₂ in FIG. 2). Because microarray data usually have a very high correlation, it is better to use a multivariate distance concept instead of the simple univariate tₖ-distance. A natural generalization of the tₖ-distance is given by Hotelling's T² statistic, defined as Equation 4:

$${T}^{2} ( i ) = {( {m}_{i} - μ )}^{′} {S}^{- 1} ( {m}_{i} - μ ) , with mean$$

$$μ = ( 1 / {N}_{C} ) {\sum }_{i = 1}^{{N}_{C}} {m}_{i} and sample covariance matrix$$

$$S = 1 / ( {N}_{C} - 1 ) {\sum }_{i = 1}^{{N}_{C}} ( {m}_{i} - μ ) {( {m}_{i} - μ )}^{′} .$$

Assuming that the mᵢ are multivariate normally distributed, T² multiplied by a constant follows a F-distribution with N_{C}-N_{CpG} degrees of freedom and the non-centrality parameter N_{CpG}. This can be used to define the upper limit of the admissible region for a given significance level α.

Two problems arise when we want to use the T²-distance for microarray data:
1. For less chips N_{C} than measurements N_{CpG}, the sample covariance matrix S is singular and not invertible.
2. The estimates for μ and S are not robust against outliers.

The first problem can be addressed by using principle component analysis (PCA) to reduce the dimensionality of our measurement space. This is done by projecting all methylation profiles mᵢ onto the first d eigenvectors with the highest variance. As a result we get the d-dimensional centered vectors i=P_{PCA}(mᵢ−μ) in eigenvector space. After the projection, the covariance matrix=diag(1, . . . , d) of the reduced space is a diagonal matrix and the T²-distance of Equation 4 is approximated by the T²-distance in the reduced space

$${\tilde{T}}^{2} ( i ) = {\sum }_{r = 1}^{d} \frac{{\tilde{m}}_{ir}^{2}}{{\tilde{s}}_{r}^{2}} .$$

Under the assumption that the true variance is equal to \tilde{S}ⱼ, \tilde{T}² follows a χ² distribution with d degrees of freedom. This can be used to define the upper significance level α. However the problem remains that the estimated eigenvectors and variances \tilde{s}ⱼ are not robust against outliers.

We propose to solve the problem of outlier sensitivity together with the dimension reduction step by using robust principle component analysis (rPCA). rPCA finds the first d directions with the largest scale in data space, robustly approximating the first d eigenvectors. The algorithm starts with centering the data with a robust location estimator. Here we will use the L₁ median according to Equation 6:

$${μ}_{L1} = {arg min}_{x} {\sum }_{i = 1}^{{N}_{C}} {‖ {m}_{i} - x ‖}_{2} .$$

In contrast to the simple component-wise median, this gives a robust estimate of the distribution center that is invariant to orthogonal linear transformations such as PCA. Then all centered observations are projected onto a finite subset of all possible directions in measurement space. The direction with maximum robust scale is chosen as an approximation of the largest eigenvector (e.g. by using the Qₙ estimator). After projecting the data into the orthogonal subspace of the selected “eigenvector” the procedure searches for an approximation of the next eigenvector. Here the finite set of possible directions is simply chosen as the set of centered observations themselves.

After obtaining the robust projection of our data into a d dimensional subspace we can compute the upper limit of the admissible region ²UCL, also referred to as the upper control limit (UCL). For a given significance level iz it is computed as Equation 7:
\tilde{T}_{UCL}²=χ_{d.1-α}.

Every observation mᵢ with T²(i)>²UCL is regarded as an outlier.

Results

In order to test how the rPCA algorithm works on microarray data we applied it to the Lymphoma dataset and compared its performance to classical PCA. The results are shown in FIG. 3.

The rPCA algorithm detected 97% of the chips with “unacceptable” quality, whereas classical PCA only detected 29%. 10% of the “acceptable” chips were detected as outliers by rPCA, whereas PCA detected 3%. rPCA detected 21 chips as outliers which were classified as “good”. These chips have all been confirmed to show saturated hybridization signals, not identified by visual inspection. This means rPCA is able to detect nearly all cases of outlier chips identified by visual inspection. Additionally rPCA detects microarrays which have unconspicous image quality but show an unusual hybridization pattern.

An obvious concern with this use of rPCA for outlier detection is that it relies on the assumption of normal distribution of the data If the distribution of the biological data is highly multi-modal, biological subclasses may be wrongly classified as outliers. To quantify this effect we simulated a very strong cluster structure in the Lymphoma data by shifting one of the smaller subclasses by a multiple of the standard deviation. Only when the measurements of all 174 CpG of the subclass where shifted by more than 2 standard deviations a considerable part of the biological samples were wrongly classified as outliers. In order to avoid such a misclassification, we tolerate at most 50% of repeated measurements of a single biological sample to be classified as outliers. However, we never reached this threshold in practice.

Statistical Process Control

Methods

In the last section we have seen how outliers can be detected solely on the basis of the overall data distribution. Statistical process control expands this approach by introducing the concept of time. The aim is to observe the variables of a process for some time under perfect working conditions. The data collected during this period form the so called historical data set (HDS), also referred to above as the ‘reference data set’. Under the assumption that all variables are normally distributed, the mean μ_{HDS} and the sample covariance matrix S_{HDS} of the historical data set fully describe the statistical behavior of the process.

Given the historical data set it becomes possible to check at any time point, I, how far the current state of the process has deviated from the perfect state by computing the T²-distance between the ideal process mean μ_{HDS} and the current observation mᵢ. This corresponds to Equation 4 with the overall sample estimates μ and S replaced by their reference counterparts μ_{HDS} and S_{HDS}. Any change in the process will cause observations with greater T²-distances. To decide whether an observation shows a significant deviation from the HDS we compute the upper control limit as in Equation 8:

$${T}_{UCL}^{2} = \frac{p ( n + 1 ) ( n - 1 )}{n ( n - p )} {F}_{p , n - p , 1 - α ,} ,$$

where p is the number of observed variables, n is the number of observations in the HDS, α is the significance level and F is the F-distribution with n-p degrees of freedom and the non-centrality parameter p. Whenever T²>T²_{UCL} is observed the process has to be regarded as significantly out of control.

In our case the process to control is a microarry experiment and the only process variables we have observed are the log ratios of the actual hybridization intensities. A single observation is then a chip mᵢ and the HDS of size N_{HDS} is defined as (m₁, . . . , m_{NHDS}}. We have to be aware of a few important issues in this interpretation of statistical process control. First, our data has a multi-modal distribution which results from a mixture of different biological samples and classes. Therefore the assumption of normality is only a rough approximation and T²_{UCL} from Equation should be regarded with caution. Secondly, as we have seen in the last sections, microarray experiments produce outliers, resulting in transgression of the UCL. This means sporadic violations of the UCL are normal and do not indicate that the process is out of control. The third issue is that we have to use the assumption that a microarray study will not systematically change its data generating distribution over time. Therefore the experimental design has to be randomized or block randomized, otherwise a systematic change in the correctly measured biological data will be interpreted as an out of control situation (e.g. when all patients with the same disease subtype are measured in one block). Finally, the question remains of what time means in the context of a microarray experiment. Beside the biological variation in the data, there are a multitude of different parameters which can systematically alter the final hybridization intensities. The experimental series should stay constant with regard to all of them. In our experience the best initial choice is to order the chips by their date of hybridization, which shows a very high correlation to most parameters of interest.

Although it is certainly interesting to look how single hybridization experiments mᵢ compare to the HDS, we are more interested in how the general behavior of the chip process changes over time. Therefore we define the current data set (CDS) (also referred to above as the test data set) as {m_{i-NCDS/2}, . . . , mᵢ, . . . , m_{i+NCDS/2}}, where i is the time of interest This allows us to look at the data distribution in a time interval of size N_{CDS} around i. In analogy to the classical setting in statistical process control we can define the T²-distance between the HDS and the CDS as in Equation 9:
T_{w}²(i)=(μ_{HDS}−μ_{CDS})^{T}^{_{S}}
where \tilde{S} is calculated from the sample covariance matrices S_{HDS} and S_{CDS} as

$$\begin{matrix}Equation 10 \text{:} & \\ \overline{S} = \frac{( {N}_{HDS} - 1 ) {S}_{HDS} + ( {N}_{CDS} - 1 ) {S}_{CDS}}{{N}_{HDS} + {N}_{CDS} - 2} . & \end{matrix}$$

Although it is possible to use T²_{w}-distance between the historical and current data set to test for μ_{HDS}=μ_{CDS}, this information is relatively meaningless. The hypothesis that the means of HDS and CDS are equal would almost always be rejected, due to the high power of the test. What is of more interest is T itself, which is the amount by which the two sample means differ in relation to the standard deviation of the data.

In order to see whether an observed change of the T²_{w}-distance comes from a simple translation it is also interesting to compare the two sample covariances S_{HDS} and S_{CDS}. A translation in log(CG/TG) space means that the hybridization intensities of HDS and CDS differ only by a constant factor (e.g. a change in probe concentration). This situation can be detected by looking at

$$L ( i ) = 2 [ ln \vert \overline{S} \vert - \frac{{N}_{HDS} - 1}{{N}_{HDS} + {N}_{CDS} - 2} ln \vert {S}_{HDS} \vert - \frac{{N}_{CDS} - 1}{{N}_{HDS} + {N}_{CDS} - 2} ln \vert {S}_{CDS} \vert ] ,$$

which is the test statistics of the likelihood ratio test for different covariance matrices. It gives a distance measure between the two covariance matrices (i.e. L=0 means equal covariances).

Before we can apply the described methods to a real microarray data set we have again to solve the problem that we need a non-singular and outlier resistant estimate of S_{HDS} and S_{CDS}. What makes the problem even harder than is that we cannot a priori know how a change in experimental conditions will affect our data. In contrast to the last section, the simple approximation of S_{HDS} by its first principle components will not work here. The reason is that changes in the experimental conditions outside the HDS will not necessarily be represented in the first principole components of S_{HDS}.

The solution is to first embed all the experimental data into a lower dimensional space by PCA. This works, because any significant change in the experimental conditions will be captured by one of the first principle components. S_{HDS} and S_{CDS} can then be reliably computed in the lower dimensional embedding. The problem of robustness is simply solved by first using robust PCA to remove outliers before performing the actual embedding and before computing the sample covariances. A summary of our algorithm is:
1. Order chips according to the parameter of interest e.g. date of hybridisation.
2. Take the set of ordered chips [m₁, . . . , m_{N}_{_{C}}] remove outliers with rPCA for computing the first d eigenvectors with classical PCA.
3. Project the set of all ordered chips {m₁, . . . , m_{N}_{_{C}}}, into the d-dimensinal subspace spanned by the computed vectors.
4. Select the first N_{HDS} chips {m₁, . . . , m_{N}_{_{HDS}}} as historical data set, remove outliers with rPCA for computing μ_{HDS} and S_{HDS}.
5. For every time index iε{1, . . . , N_{C}}
(a) Compute T² distance between mᵢ and μ_{HDS}.

$$( b ) If \frac{{N}_{CDS}}{2} < i < {N}_{C} - \frac{{N}_{CDS}}{2}$$

$$i . Select \{ {m}_{i} - {N}_{CDS} / 2 , … , {m}_{i} , … , {m}_{i} + {N}_{CDS} / 2 \} as current$$

$$data set , remove outliers with rPCA for computing {μ}_{CDS}$$

$$and {S}_{CDS} . \\ ii . Compute {T}_{w}^{2} - distance between {μ}_{HDS} and {μ}_{CDS} . \\ iii . Compute L - distance between {S}_{HDS} and {S}_{CDS} .$$
6. Generate controlling chart by plotting T². T_{w}² and L

With the computed values for T², T²_{w} and L we can generate a plot that visualizes the quality development of the chip process over time, a so called T² control chart.

Results

The first example is shown in FIG. 4, which demonstrates how our algorithm detects a change in hybridization temperature. As can be expected the T²-value grows with an increase in hybridization temperature. The systematic increase of the L-distance indicates that this is not only caused by a simple translation in methylation space. The process has to be regarded as clearly out of control, due to the observation that almost all chips are above the UCL after the temperature change and the process center has drifted more than T_{w}=4 standard deviations away from its original location.

FIG. 6 shows how our method detects the simulated handling error in the Lymphoma data set The affected chips can be clearly identified by the significant increase in the T²-distances as well as by their change in the covariance structure.

Finally, FIG. 5 shows the T² control chart of the ALL/AML study. It clearly indicates that the experimental conditions significantly changed two times over the course of the study. A look at the L-distance reveals that the covariance within the two detected artefact blocks is identical to the HDS. A change in covariance can be detected only when the CDS window passes the two borders. This clearly indicates that the observed effect is a simple translation of the process mean.

The major practical problem is now to identify the reasons for the changes. In this regard the most valuable information from the T² control chart is the time point of process change. It can be cross-checked with the laboratory protocol and the process parameters which have changed at the same time can be identified. In our case the two process shifts corresponded to the time of replacement of re-synthesized probe oligos for slide production, which were obviously delivered at a wrong concentration. After exclusion of the affected CpG positions from the analysis the T² chart showed normal behavior and the overall noise level of the data set was significantly reduced.

Discussion

Taken together, we have shown that robust principle components analysis and techniques of statistical process control can be used to detect flaws in microarray experiments. Robust PCA has proven to be able to automatically detect nearly all cases of outlier chips identified by visual inspection; as well as microarrays with unconspicous image quality but saturated hybridization signals. With the T² control chart we introduced a tool that facilitates the detection and assessment of even minor systematic changes in large scale microarray studies.

A major advantage of both methods is that they do not rely on an explicit modeling of the microarray process as they are solely based on the distribution of the actual measurements. Having successfully applied our methods to the example of DNA methylation data, we assume that the same results can be achieved with other types of microarray platforms. The sensitivity of the methods improve with increasing study sizes, due to their multivariate nature. This makes them particularly suitable for medium to large scale experiments in a high throughput environment.

The retrospective analysis of a study with our methods can greatly improve results and avoid misleading biological interpretations. When the T² control chart is monitored in real time a given quality level can be maintained in a very cost effective way. On the one hand, this allows for an immediate correction of process parameters. On the other hand, this makes it possible to specifically repeat only those slides affected by a process artefact This guarantees high quality while minimizing the number of repetitions.

A general shortcoming of T² control charts is that they only indicate that something went wrong, but not what was exactly the source. Therefore we have used the time at which a significant change happened in order to identify the responsible process parameter. We have shown how a quantification of the change in covariance structure provides additional information and permits to discriminate between different problems like changes in probe concentration and accidental handling errors.

### Example 2

In one aspect, the method according to the disclosed invention provides a means for automatically generating a concise report based on the disclosed methods for quality monitoring of laboratory process performance. In the disclosed embodiment this report is structured in sections starting with summary table (see Table 1) of the performance grades for several evaluation categories of the individual experiment units, a section detailing each evaluation category in turn in a table of grades for this category, the corresponding performance variables the grades are based on and a set of graphical displays implemented as panel of box plots (see FIG. 7) displaying the thresholds used for grading, and a table of details containing all evaluation grades for each experimental unit. The report can be generated by means of a computer program which outputs the result in file formats HTML, Adobe PDF, postscript, and variants thereof

TABLE 1

| | | Rob. | | | | |
|---|---|---|---|---|---|---|
| | Vis. | PCA - | | | | |
| Chip | Grade | Thr. | BG | SPOT | GEO | SAT |
| 0100870030-68406- | 3 | −0.9 | bad | good | bad | good |
| 57115 | | | | | | |
| 0100870296-68421- | 2 | −1.5 | bad | good | bad | good |
| 57110 | | | | | | |
| 0100870569-68422- | 2 | −2.7 | bad | good | bad | good |
| 57121 | | | | | | |
| 0100870907-68447- | 2 | −2. | bad | good | bad | good |
| 57105 | | | | | | |
| 0100870949-68451- | 2 | −1.8 | dubious | good | bad | good |
| 57127 | | | | | | |
| 0100871228-68460- | 2 | −1.9 | dubious | good | bad | good |
| 57104 | | | | | | |
| 0l00871947-68487- | 1 | −1.6 | dubious | good | bad | good |
| 57109 | | | | | | |
| 0100871997-68491- | 2 | −2.1 | bad | good | bad | good |
| 57128 | | | | | | |
| 0100872531-68503- | 6 | 5.6 | bad | good | good | good |
| 57103 | | | | | | |
| 0100872549-68495- | 1 | 2.3 | bad | good | bad | good |
| 57112 | | | | | | |
| 0100872573-68504- | 2 | −0.2 | bad | good | bad | good |
| 57129 | | | | | | |
| 0100872812-68517- | 2 | −1.4 | dubious | good | bad | good |
| 57106 | | | | | | |
| 0100870056-68408- | 3 | −1.8 | bad | good | bad | good |
| 57133 | | | | | | |
| 0100870072-68410- | 3 | −2.1 | bad | good | bad | good |
| 57139 | | | | | | |

Table 1 shows the summary table of category grades for each experimental unit: From left to right, the columns represent the identifier of the experimental unit, the human expert visual grade, the distance for the experimental unit from the estimate the robust mean location of the set of experiments, the background category grade, the spot characteristic category grade, the geometry characteristic grade and the intensity saturation category grade are stated. Three grade levels are used, good, dubious, bad, based on the grades calculated for each category in turn.

Table 2 shows the complete summary table of all chips analysed in study ‘1’ according to FIG. 7, of which Table 1 represents the most informative subset

TABLE 2

| | | Rob. | | | | |
|---|---|---|---|---|---|---|
| | vis. | PCA - | | | | |
| Chip | Grade | Thr. | BG | SPOT | GEO | SAT |
| 0100870030- | 3 | −0.9 | bad | good | bad | good |
| 68406-57115 | | | | | | |
| 0100870296- | 2 | −1.5 | bad | good | bad | good |
| 68421-57110 | | | | | | |
| 0100870569- | 2 | −2.7 | bad | good | bad | good |
| 68422-57121 | | | | | | |
| 0100870907- | 2 | −2 | dubious | good | bad | good |
| 68447-57105 | | | | | | |
| 0100870949- | 2 | −1.8 | dubious | good | bad | good |
| 68451-57127 | | | | | | |
| 0100871228- | 2 | −1.9 | dubious | good | bad | good |
| 68460-57104 | | | | | | |
| 0100871947- | 1 | −1.6 | dubious | good | bad | good |
| 68487-57109 | | | | | | |
| 0100871997- | 2 | −2.1 | bad | good | bad | good |
| 68491-57128 | | | | | | |
| 0100872531- | 6 | 5.6 | bad | good | good | good |
| 68503-57103 | | | | | | |
| 0100872549- | 1 | 2.3 | bad | good | bad | good |
| 68495-57112 | | | | | | |
| 0100872573- | 2 | −0.2 | bad | good | bad | good |
| 68504-57129 | | | | | | |
| 0100872812- | 2 | −1.4 | dubious | good | bad | good |
| 68517-57106 | | | | | | |
| 0100870056- | 3 | −1.8 | bad | good | bad | good |
| 68408-57133 | | | | | | |
| 0100870072- | 3 | −2.1 | bad | good | bad | good |
| 68410-57139 | | | | | | |
| 0100870098- | 3 | −1.2 | good | good | bad | good |
| 68412-57145 | | | | | | |
| 0100870171- | 3 | −1.3 | good | good | bad | good |
| 68417-57183 | | | | | | |
| 0100870402- | 2 | −2.2 | dubious | good | bad | good |
| 68426-57164 | | | | | | |
| 0100870527- | 2 | −2.6 | bad | good | bad | good |
| 68437-57107 | | | | | | |
| 0100870600- | 2 | −0.8 | bad | good | bad | good |
| 68439-57146 | | | | | | |
| 0100870642- | 3 | −1.5 | bad | good | bad | good |
| 68442-57165 | | | | | | |
| 0100870725- | 2 | −0.7 | bad | good | good | good |
| 68444-57185 | | | | | | |
| 0100870923- | 3 | −2.5 | dubious | good | bad | good |
| 68449-57117 | | | | | | |
| 0100870965- | 2 | −1 | dubious | good | bad | good |
| 68453-57140 | | | | | | |
| 0100870981- | 2 | −1.5 | dubious | good | bad | good |
| 68438-57143 | | | | | | |
| 0100871004- | 2 | −1.8 | dubious | good | bad | good |
| 68441-57153 | | | | | | |
| 0100871020- | 2 | −2.4 | good | good | bad | good |
| 68455-57166 | | | | | | |
| 0100871046- | 2 | −1.9 | bad | good | bad | good |
| 68443-57172 | | | | | | |
| 0100871062- | 2 | −1.1 | bad | good | bad | good |
| 68445-57180 | | | | | | |
| 0100871301- | 2 | −1.8 | good | good | bad | good |
| 68464-57141 | | | | | | |
| 0100871343- | 2 | −1.5 | dubious | good | bad | good |
| 68467-57160 | | | | | | |
| 0100871632- | 2 | −2.1 | bad | good | bad | good |
| 68478-57119 | | | | | | |
| 0100871674- | 2 | −2 | dubious | good | bad | good |
| 68468-57136 | | | | | | |
| 0100871757- | 3 | −1.9 | bad | good | bad | good |
| 68470-57157 | | | | | | |
| 0100871799- | 2 | −0.7 | bad | good | bad | good |
| 68482-57167 | | | | | | |
| 0100871822- | 3 | −2.1 | good | good | bad | good |
| 68483-57176 | | | | | | |
| 0100871830- | 3 | −1.2 | dubious | good | bad | good |
| 68472-57179 | | | | | | |
| 0100872185- | 3 | −0.1 | bad | good | bad | good |
| 68484-57138 | | | | | | |
| 0100872226- | 3 | −0.8 | dubious | good | bad | good |
| 68492-57149 | | | | | | |
| 0100872268- | 3 | −2.1 | dubious | good | bad | good |
| 68494-57154 | | | | | | |
| 0100872309- | 2 | −2 | dubious | good | bad | good |
| 68494-57168 | | | | | | |
| 0100872341- | 2 | −1.5 | dubious | good | bad | good |
| 68488-57174 | | | | | | |
| 0100872383- | 2 | −1.1 | bad | good | dubious | good |
| 68496-57187 | | | | | | |
| 0100872581- | 2 | −0.6 | bad | good | bad | good |
| 68506-57142 | | | | | | |
| 0100872614- | 2 | −2 | bad | good | bad | good |
| 68508-57150 | | | | | | |
| 0100872622- | 2 | −1.4 | bad | good | bad | good |
| 68498-57152 | | | | | | |
| 0100872656- | 2 | −2.7 | bad | good | bad | good |
| 68510-57169 | | | | | | |
| 0100872664- | 2 | −1.9 | bad | good | bad | good |
| 68512-57175 | | | | | | |
| 0100872698- | 2 | −2 | bad | good | bad | good |
| 68500-57181 | | | | | | |
| 0100872820- | 2 | −1.2 | bad | good | bad | good |
| 68509-57113 | | | | | | |
| 0100872854- | 2 | −1.9 | bad | good | bad | good |
| 68511-57132 | | | | | | |
| 0100872896- | 2 | 2.7 | bad | good | bad | good |
| 68514-57137 | | | | | | |
| 0100872903- | 2 | −2 | bad | good | bad | good |
| 68519-57151 | | | | | | |
| 0100872937- | 2 | −1.9 | bad | good | bad | good |
| 68516-57155 | | | | | | |
| 0100872979- | 2 | −0.8 | dubious | good | bad | good |
| 68521-57178 | | | | | | |
| 0100873068- | 3 | −2.9 | dubious | good | bad | good |
| 68405-57182 | | | | | | |
| 0100870212- | 3 | −1.7 | bad | good | bad | good |
| 68403-57198 | | | | | | |
| 0100870246- | 3 | −0.4 | dubious | good | bad | good |
| 68559-57265 | | | | | | |
| 0100870254- | 3 | −1.7 | bad | good | good | good |
| 68404-57216 | | | | | | |
| 0100870288- | 2 | −2.2 | good | good | bad | good |
| 68527-57233 | | | | | | |
| 0100870329- | 3 | −0.3 | bad | good | bad | good |
| 68529-57235 | | | | | | |
| 0100870361- | 3 | −1.5 | dubious | good | bad | good |
| 68555-57261 | | | | | | |
| 0100870444- | 3 | −1 | bad | good | bad | good |
| 68432-57195 | | | | | | |
| 0l00870452- | 3 | 1.9 | bad | good | bad | good |
| 68433-57204 | | | | | | |
| 0100870486- | 2 | −2.7 | dubious | good | bad | good |
| 68418-57215 | | | | | | |
| 0100870759- | 3 | −2.2 | bad | good | bad | good |
| 68528-57234 | | | | | | |
| 0100870767- | 2 | −2.1 | dubious | good | bad | good |
| 68429-57191 | | | | | | |
| 0100870791- | 3 | −2 | bad | good | bad | good |
| 68531-57237 | | | | | | |
| 0100870808- | 2 | −0.8 | dubious | good | bad | good |
| 68431-57197 | | | | | | |
| 0100870832- | 3 | −1.3 | dubious | good | bad | good |
| 68533-57239 | | | | | | |
| 0100870840- | 2 | −1.5 | dubious | good | bad | good |
| 68434-57208 | | | | | | |
| 0100870866- | 2 | −3.2 | dubious | good | bad | good |
| 68446-57220 | | | | | | |
| 0100870882- | 2 | −2.1 | bad | good | bad | good |
| 68436-57223 | | | | | | |
| 0100870915- | 2 | −1.3 | dubious | good | bad | good |
| 68536-57242 | | | | | | |
| 0100870957- | 3 | 3.2 | bad | good | bad | good |
| 68532-57238 | | | | | | |
| 0100870999- | 3 | −1.2 | bad | good | bad | good |
| 68538-57244 | | | | | | |
| 0100871070- | 3 | −2.3 | dubious | good | bad | good |
| 68543-57249 | | | | | | |
| 0100871088- | 3 | −0.8 | dubious | good | bad | good |
| 68448-57190 | | | | | | |
| 0100871103- | 4 | −1.2 | bad | good | good | good |
| 68450-57199 | | | | | | |
| 0100871129- | 3 | −2.5 | bad | good | bad | good |
| 68452-57210 | | | | | | |
| 0100871145- | 3 | −1.9 | bad | good | bad | good |
| 68535-57241 | | | | | | |
| 0100871161- | 2 | −1.5 | bad | good | bad | good |
| 68457-57221 | | | | | | |
| 0100871187- | 2 | −2.2 | dubious | good | bad | good |
| 68547-57253 | | | | | | |
| 0100871195- | 2 | −0.8 | good | good | bad | good |
| 68550-57256 | | | | | | |
| 0100871236- | 3 | −0.3 | bad | good | dubious | good |
| 68537-57266 | | | | | | |
| 0100871260- | 2 | −2.4 | dubious | good | bad | good |
| 68552-57258 | | | | | | |
| 0100871278- | 3 | −1.1 | dubious | good | bad | good |
| 68539-57245 | | | | | | |
| 0100871335- | 2 | −0.9 | dubious | good | bad | good |
| 68541-57247 | | | | | | |
| 0100871385- | 4 | 0.5 | bad | good | good | good |
| 68542-57248 | | | | | | |
| 0100871468- | 4 | −2.2 | good | good | bad | good |
| 68461-57200 | | | | | | |
| 0100871476- | 2 | −1.5 | bad | good | bad | good |
| 68548-57254 | | | | | | |
| 0100871517- | 2 | −3.1 | dubious | good | bad | good |
| 68558-57264 | | | | | | |
| 0100871559- | 3 | −2.7 | dubious | good | bad | good |
| 68463-57217 | | | | | | |
| 0100871591- | 2 | −2.1 | dubious | good | bad | good |
| 68475-57228 | | | | | | |
| 0100871864- | 2 | −2.4 | bad | good | bad | good |
| 68485-57196 | | | | | | |
| 0100871872- | 2 | −1.1 | bad | good | bad | good |
| 68474-57201 | | | | | | |
| 0100871898- | 2 | −2.5 | dubious | good | bad | good |
| 68477-57209 | | | | | | |
| 0100871905- | 3 | −2.6 | bad | good | bad | good |
| 68479-57218 | | | | | | |
| 0100871913- | 2 | −0.7 | dubious | good | bad | good |
| 68481-57225 | | | | | | |
| 0100872101- | 3 | −2.4 | dubious | good | bad | good |
| 68551-57257 | | | | | | |
| 0100872143- | 3 | −2.1 | bad | good | good | good |
| 68553-57259 | | | | | | |
| 0100872424- | 3 | −1 | dubious | good | bad | good |
| 68490-57188 | | | | | | |
| 0100872458- | 3 | −1.9 | bad | good | bad | good |
| 68497-57205 | | | | | | |
| 0100872466- | 2 | −0.9 | bad | good | bad | good |
| 68499-57213 | | | | | | |
| 0100872490- | 2 | −0.7 | bad | good | bad | good |
| 68493-57219 | | | | | | |
| 0100872507- | 3 | −0.9 | bad | good | bad | good |
| 68501-57229 | | | | | | |
| 0100872705- | 3 | −2.5 | bad | good | bad | good |
| 68502-57193 | | | | | | |
| 0100872739- | 2 | −2 | bad | good | bad | good |
| 68505-57202 | | | | | | |
| 0100872747- | 2 | −0.7 | bad | good | bad | good |
| 68513-57214 | | | | | | |
| 0100872771- | 2 | −1.8 | dubious | good | bad | good |
| 68515-57222 | | | | | | |
| 0100872789- | 2 | −0.2 | bad | good | bad | good |
| 68524-57230 | | | | | | |
| 0100872862- | 2 | −0.5 | bad | good | bad | good |
| 68560-57267 | | | | | | |
| 0100872987- | 2 | −3.1 | bad | good | bad | good |
| 68526-57232 | | | | | | |
| 0100873183- | 4 | −2.2 | bad | good | bad | good |
| 68401-57207 | | | | | | |
| 0100870022- | 3 | −1.2 | good | good | bad | good |
| 68703-57410 | | | | | | |
| 0100870048- | 5 | −0.8 | bad | good | bad | good |
| 68704-57411 | | | | | | |
| 0100870080- | 3 | −2.6 | dubious | good | bad | good |
| 68562-57271 | | | | | | |
| 0100870105- | 3 | −0.9 | bad | good | bad | good |
| 68701-57408 | | | | | | |
| 0100870121- | 3 | −1.5 | dubious | good | bad | good |
| 68564-57273 | | | | | | |
| 0100870147- | 3 | −1.3 | bad | good | bad | good |
| 68699-57406 | | | | | | |
| 0100870163- | 4 | −0.8 | dubious | good | bad | bad |
| 68563-57269 | | | | | | |
| 0l00870l89- | 3 | 0.4 | bad | good | bad | good |
| 68700-57407 | | | | | | |
| 0100870204- | 3 | −1.1 | bad | good | bad | good |
| 68565-57268 | | | | | | |
| 0100870775- | 3 | −2.7 | dubious | good | bad | good |
| 68696-57403 | | | | | | |
| 0100870816- | 3 | −0.7 | bad | good | bad | good |
| 68698-57405 | | | | | | |
| 0100870858- | 3 | −1.7 | dubious | good | bad | good |
| 68697-57404 | | | | | | |
| 0100870890- | 3 | −0.7 | bad | good | bad | good |
| 68575-57281 | | | | | | |
| 0100870931- | 3 | 1 | bad | good | good | good |
| 68576-57283 | | | | | | |
| 0100871012- | 3 | −1.6 | dubious | good | bad | good |
| 68691-57398 | | | | | | |
| 0100871054- | 3 | −2.2 | bad | good | bad | good |
| 68692-57399 | | | | | | |
| 0100871096- | 2 | −0.6 | good | good | bad | good |
| 68638-57345 | | | | | | |
| 0100871137- | 2 | −2.4 | bad | good | bad | good |
| 68636-57343 | | | | | | |
| 0100871179- | 5 | −0.9 | dubious | dubious | bad | good |
| 68650-57357 | | | | | | |
| 0100871210- | 3 | −1.6 | bad | good | bad | good |
| 68706-57413 | | | | | | |
| 0100871252- | 3 | −2 | dubious | good | bad | good |
| 68649-57356 | | | | | | |
| 0100871294- | 3 | −1 | bad | good | bad | good |
| 68635-57342 | | | | | | |
| 0100871418- | 2 | −1.2 | bad | good | bad | good |
| 68615-57322 | | | | | | |
| 0100871450- | 2 | −2 | dubious | good | bad | good |
| 68678-57385 | | | | | | |
| 0100871492- | 2 | −0.7 | dubious | good | bad | good |
| 68677-57384 | | | | | | |
| 0100871533- | 5 | −2.1 | dubious | good | bad | good |
| 68676-57383 | | | | | | |
| 0100871541- | 3 | −0.4 | dubious | good | bad | good |
| 68645-57352 | | | | | | |
| 0100871583- | 3 | −1.7 | bad | good | bad | good |
| 68643-57350 | | | | | | |
| 0100871624- | 2 | −2.3 | dubious | good | bad | good |
| 68644-57351 | | | | | | |
| 0100871666- | 5 | −1.5 | bad | good | bad | good |
| 68642-57349 | | | | | | |
| 0100871707- | 2 | −2.9 | dubious | good | bad | good |
| 68641-57348 | | | | | | |
| 0100871731- | 3 | −3.4 | bad | good | bad | good |
| 68571-57277 | | | | | | |
| 0100871773- | 2 | 3.6 | bad | good | bad | good |
| 68572-57278 | | | | | | |
| 0100871781- | 4 | −1.7 | bad | good | bad | good |
| 68675-57382 | | | | | | |
| 0100871814- | 3 | −0.7 | bad | good | bad | good |
| 68573-57282 | | | | | | |
| 0100871856- | 2 | −0.1 | bad | good | bad | good |
| 68574-57280 | | | | | | |
| 0100871939- | 2 | −2 | bad | good | bad | good |
| 68561-57270 | | | | | | |
| 0100871971- | 4 | −1.7 | bad | good | bad | good |
| 68569-57276 | | | | | | |
| 0100871989- | 2 | −2.3 | bad | good | bad | good |
| 68570-57279 | | | | | | |
| 0100872135- | 3 | −2.3 | dubious | good | bad | good |
| 68651-57358 | | | | | | |
| 0100872177- | 3 | −1.4 | bad | good | bad | good |
| 68652-57359 | | | | | | |
| 0100872218- | 4 | −2.3 | dubious | dubious | bad | good |
| 68653-57360 | | | | | | |
| 0100872250- | 4 | −0.6 | good | good | bad | good |
| 68654-57361 | | | | | | |
| 0100872292- | 3 | −2.2 | dubious | good | bad | good |
| 68655-57362 | | | | | | |
| 0100872333- | 4 | −0.5 | bad | dubious | bad | good |
| 68656-57363 | | | | | | |
| 0100872375- | 3 | −0.6 | good | good | bad | good |
| 68656-57397 | | | | | | |
| 0100872416- | 3 | −1.3 | bad | good | bad | good |
| 68689-57396 | | | | | | |
| 0100873018- | 3 | −1.3 | bad | good | bad | good |
| 68601-57308 | | | | | | |
| 0100873026- | 3 | −2.4 | dubious | good | bad | good |
| 68602-57309 | | | | | | |
| 0100873050- | 5 | −1.5 | bad | good | bad | good |
| 68659-57366 | | | | | | |
| 0100873076- | 3 | −1.2 | bad | good | bad | good |
| 68578-57285 | | | | | | |
| 0100873084- | 5 | −1.4 | bad | good | bad | good |
| 68664-57371 | | | | | | |
| 0100873117- | 3 | −1.6 | bad | good | bad | good |
| 68581-57288 | | | | | | |
| 0100873133- | 3 | −1.8 | bad | good | bad | good |
| 68679-57386 | | | | | | |
| 0100873159- | 3 | −1.6 | bad | good | bad | good |
| 68580-57287 | | | | | | |
| 0100873175- | 2 | −2.4 | bad | good | bad | good |
| 68681-57388 | | | | | | |
| 0100873191- | 2 | −1.4 | bad | good | bad | good |
| 68630-57337 | | | | | | |
| 0100873216- | 2 | −2.3 | bad | good | bad | good |
| 68682-57389 | | | | | | |
| 0100873224- | 3 | −1.6 | dubious | good | bad | good |
| 68627-57334 | | | | | | |
| 0100873232- | 2 | −0.3 | bad | good | bad | good |
| 68629-57336 | | | | | | |
| 0100873258- | 2 | −2.1 | dubious | good | bad | good |
| 68684-57391 | | | | | | |
| 0100873266- | 3 | −1.3 | bad | good | bad | good |
| 68628-57335 | | | | | | |
| 0100873274- | 3 | −1.1 | bad | good | bad | good |
| 68631-57338 | | | | | | |
| 0100873290- | 3 | −2 | dubious | good | bad | good |
| 68683-57390 | | | | | | |
| 0100873307- | 3 | −1.8 | bad | good | bad | good |
| 68625-57332 | | | | | | |
| 0100873315- | 2 | −0.4 | bad | good | bad | good |
| 68586-57293 | | | | | | |
| 0100873331- | 3 | −1.9 | bad | good | bad | good |
| 68686-57393 | | | | | | |
| 0100873349- | 4 | −2.3 | dubious | good | bad | good |
| 68626-57333 | | | | | | |
| 0100873357- | 2 | −1.3 | bad | good | bad | good |
| 68585-57292 | | | | | | |
| 0100873373- | 5 | −1.5 | bad | dubious | bad | good |
| 68685-57392 | | | | | | |
| 0100873381- | 2 | −2.7 | bad | good | bad | good |
| 68639-57346 | | | | | | |
| 0100873399- | 3 | −1.8 | bad | good | bad | good |
| 68589-57296 | | | | | | |
| 0100873414- | 2 | −0.4 | bad | good | dubious | good |
| 68687-57394 | | | | | | |
| 0100873422- | 2 | −2.2 | bad | good | bad | good |
| 68624-57331 | | | | | | |
| 0100873430- | 3 | −1.1 | bad | good | bad | good |
| 68587-57294 | | | | | | |
| 0100873456- | 3 | −1.3 | bad | good | bad | good |
| 68688-57395 | | | | | | |
| 0100873464- | 3 | −1.6 | dubious | good | bad | good |
| 68666-57373 | | | | | | |
| 0100873472- | 6 | −2 | dubious | good | bad | good |
| 68640-57347 | | | | | | |
| 0100873498- | 2 | −2.5 | bad | good | bad | good |
| 68665-57372 | | | | | | |
| 0100873505- | 2 | −2.2 | bad | good | bad | good |
| 68667-57374 | | | | | | |
| 0100873513- | 3 | −1.2 | good | good | bad | good |
| 68588-57295 | | | | | | |
| 0100873539- | 2 | −1.6 | bad | good | bad | good |
| 68596-57303 | | | | | | |
| 0100873547- | 3 | −1.3 | bad | good | bad | good |
| 68647-57354 | | | | | | |
| 0100873555- | 4 | −0.7 | bad | good | bad | good |
| 68590-57297 | | | | | | |
| 0100873571- | 2 | −1 | bad | good | bad | good |
| 68598-57305 | | | | | | |
| 0100873589- | 5 | −2.3 | bad | good | bad | good |
| 68648-57355 | | | | | | |
| 0100873612- | 3 | 0 | bad | good | bad | good |
| 68597-57304 | | | | | | |
| 0100873646- | 3 | −0.4 | bad | good | bad | good |
| 68595-57302 | | | | | | |
| 0100873654- | 4 | −2.7 | bad | good | bad | good |
| 68600-57307 | | | | | | |
| 0100873662- | 2 | −1.8 | dubious | good | bad | good |
| 68669-57376 | | | | | | |
| 0100873696- | 2 | −2.1 | bad | good | bad | good |
| 68599-57306 | | | | | | |
| 0100873703- | 2 | −1.7 | dubious | good | bad | good |
| 68670-57377 | | | | | | |
| 0100873737- | 4 | −1.4 | bad | good | bad | good |
| 68582-57289 | | | | | | |
| 0100873745- | 4 | −0.8 | dubious | good | bad | good |
| 68671-57378 | | | | | | |
| 0100873779- | 3 | −2.6 | bad | good | bad | good |
| 68583-57290 | | | | | | |
| 0100873787- | 5 | −2 | bad | good | bad | good |
| 68672-57379 | | | | | | |
| 0100873810- | 3 | −1.8 | dubious | good | bad | good |
| 68584-57291 | | | | | | |
| 0100873828- | 2 | −1.8 | dubious | good | bad | good |
| 68657-57364 | | | | | | |
| 0100873852- | 3 | −2.2 | bad | good | bad | good |
| 68607-57314 | | | | | | |
| 0100873860- | 2 | −1.1 | bad | good | dubious | good |
| 68662-57418 | | | | | | |
| 0100873894- | 3 | −2.6 | dubious | good | bad | good |
| 68605-57312 | | | | | | |
| 0100873901- | 2 | −1.9 | bad | good | bad | good |
| 68637-57344 | | | | | | |
| 0100873935- | 2 | −0.8 | dubious | good | bad | good |
| 68606-57313 | | | | | | |
| 0100873943- | 3 | −2.2 | bad | good | bad | good |
| 68577-57284 | | | | | | |
| 0100873969- | 3 | −0.8 | dubious | good | bad | good |
| 68661-57368 | | | | | | |
| 0100873977 | 3 | −1.8 | bad | good | bad | good |
| 68604-57311 | | | | | | |
| 0100873985- | 2 | −1.9 | bad | good | bad | good |
| 68591-57298 | | | | | | |
| 0100874008- | 2 | −1.7 | bad | good | bad | good |
| 68663-57370 | | | | | | |
| 0100874016- | 3 | −2.3 | bad | good | bad | good |
| 68603-57310 | | | | | | |
| 0100874024- | 4 | −1.1 | bad | good | bad | good |
| 68579-57286 | | | | | | |
| 0100874040- | 2 | −0.7 | bad | good | bad | good |
| 68673-57380 | | | | | | |
| 0100875147- | 4 | −1.8 | bad | good | bad | good |
| 68717-57426 | | | | | | |
| 0100875345- | 3 | −1.8 | bad | good | bad | good |
| 68719-57428 | | | | | | |
| 0100875387- | 3 | −1.6 | dubious | good | bad | good |
| 68720-57429 | | | | | | |
| 0100875428- | 3 | −0.1 | dubious | good | bad | good |
| 68716-57425 | | | | | | |
| 0100874157- | 2 | −1.5 | bad | good | bad | good |
| 68787-57518 | | | | | | |
| 0100874404- | 3 | 3.3 | bad | good | bad | good |
| 68773-57504 | | | | | | |
| 0100874446- | 3 | −2.5 | bad | good | bad | good |
| 68771-57502 | | | | | | |
| 0100874488- | 2 | −1.5 | bad | good | bad | good |
| 68800-57531 | | | | | | |
| 0100874529- | 2 | 0 | bad | good | bad | good |
| 68796-57527 | | | | | | |
| 0100874553- | 2 | −1.9 | bad | good | bad | good |
| 68792-57523 | | | | | | |
| 0100874561- | 3 | −1.6 | bad | good | bad | good |
| 68798-57529 | | | | | | |
| 0100874595- | 2 | −0.5 | bad | good | bad | good |
| 68794-57525 | | | | | | |
| 0100874602- | 3 | −1.4 | bad | good | bad | good |
| 68775-57506 | | | | | | |
| 0100874628- | 3 | −2.7 | bad | good | bad | good |
| 68808-57543 | | | | | | |
| 0100874636- | 2 | −0.9 | bad | good | bad | good |
| 68788-57519 | | | | | | |
| 0100874678- | 3 | −2.3 | bad | good | bad | good |
| 68791-57522 | | | | | | |
| 0100875098- | 2 | −1.8 | bad | good | bad | good |
| 68721-57431 | | | | | | |
| 0100875121- | 2 | −1.2 | bad | good | bad | good |
| 68735-57451 | | | | | | |
| 0100875139- | 2 | −1 | bad | good | bad | good |
| 68723-57443 | | | | | | |
| 0100875163- | 2 | −1.3 | bad | good | bad | good |
| 68733-57454 | | | | | | |
| 0100875171- | 2 | −1.9 | bad | good | bad | good |
| 68768-57499 | | | | | | |
| 0100875204- | 2 | 3.8 | bad | good | bad | good |
| 68732-57452 | | | | | | |
| 0100875212- | 3 | −2.5 | bad | good | bad | good |
| 68767-57498 | | | | | | |
| 0100875246- | 2 | −1.2 | bad | good | bad | good |
| 68730-57453 | | | | | | |
| 0100875254- | 2 | −2.5 | bad | good | bad | good |
| 68765-57489 | | | | | | |
| 0100875288- | 2 | −2.1 | bad | good | bad | good |
| 68728-57448 | | | | | | |
| 0100875296- | 4 | −2.2 | dubious | good | bad | good |
| 68815-57550 | | | | | | |
| 0100875379- | 3 | −1.7 | dubious | good | bad | good |
| 68763-57482 | | | | | | |
| 0100875410- | 3 | −2.9 | good | good | bad | good |
| 68762-57481 | | | | | | |
| 0100875452- | 3 | −1.2 | good | good | bad | good |
| 68810-57544 | | | | | | |
| 0100875494- | 2 | −2.1 | dubious | good | bad | good |
| 68759-57478 | | | | | | |
| 0100875535- | 2 | −1.7 | bad | good | bad | good |
| 68811-57545 | | | | | | |
| 0100875577- | 3 | −2.2 | dubious | good | bad | good |
| 68814-57547 | | | | | | |
| 0100875593- | 3 | −1.9 | bad | good | bad | good |
| 68785-57516 | | | | | | |
| 0100875618- | 2 | −1.7 | dubious | good | bad | good |
| 68756-57475 | | | | | | |
| 0100875759- | 3 | −0.8 | bad | good | bad | good |
| 68776-57507 | | | | | | |
| 0100875791- | 2 | −3.3 | bad | good | bad | good |
| 68774-57505 | | | | | | |
| 0100875816- | 3 | −0.3 | bad | good | bad | good |
| 68738-57457 | | | | | | |
| 0100875824- | 3 | −1.1 | dubious | good | bad | good |
| 68769-57500 | | | | | | |
| 0100875832- | 2 | −2.4 | dubious | good | bad | good |
| 68772-57503 | | | | | | |
| 0100875858- | 2 | −1.5 | bad | good | bad | good |
| 68739-57458 | | | | | | |
| 0100875866- | 2 | −1.3 | bad | good | bad | good |
| 68726-57446 | | | | | | |
| 0100875915- | 3 | −1.4 | dubious | good | bad | good |
| 68742-57461 | | | | | | |
| 0100875957- | 3 | −2 | bad | good | bad | good |
| 68741-57460 | | | | | | |
| 0100875999- | 3 | −1.5 | bad | good | bad | good |
| 68740-57459 | | | | | | |
| 0100876012- | 3 | −0.1 | bad | good | bad | good |
| 68737-57456 | | | | | | |
| 0100876038- | 2 | −2.3 | bad | good | bad | good |
| 68755-57474 | | | | | | |
| 0100876054- | 3 | −1.5 | bad | good | bad | good |
| 68736-57455 | | | | | | |
| 0100876070- | 2 | −2 | bad | good | bad | good |
| 68813-57546 | | | | | | |
| 0100876096- | 3 | −2.3 | bad | good | bad | good |
| 68784-57515 | | | | | | |
| 0100876103- | 3 | 0.2 | bad | good | bad | good |
| 68745-57464 | | | | | | |
| 0100876137- | 3 | −2.1 | bad | good | bad | good |
| 68786-57517 | | | | | | |
| 0100876145- | 3 | −0.7 | bad | good | bad | good |
| 68746-57465 | | | | | | |
| 0100876179- | 2 | −1.7 | bad | good | bad | good |
| 68780-57511 | | | | | | |
| 0100876187- | 3 | −1.3 | bad | good | bad | good |
| 68747-57466 | | | | | | |
| 0100876210- | 3 | −1.1 | bad | good | bad | good |
| 68812-57549 | | | | | | |
| 0100876228- | 3 | −1.9 | bad | good | bad | good |
| 68748-57467 | | | | | | |
| 0100876252- | 3 | −1.6 | bad | good | bad | good |
| 68777-57508 | | | | | | |
| 0100876260- | 3 | −1.6 | bad | good | bad | good |
| 68749-57468 | | | | | | |
| 0100876301- | 3 | −0.8 | dubious | good | bad | good |
| 68750-57469 | | | | | | |
| 0100876335- | 3 | −3.4 | bad | good | bad | good |
| 68790-57521 | | | | | | |
| 0100876343- | 3 | −1 | bad | good | bad | good |
| 68754-57473 | | | | | | |
| 0100876377- | 3 | −1.2 | bad | good | bad | good |
| 68793-57524 | | | | | | |
| 0100876418- | 3 | −3.2 | bad | good | bad | good |
| 68795-57526 | | | | | | |
| 0100876450- | 3 | −2.2 | bad | good | bad | good |
| 68797-57528 | | | | | | |
| 0100876492- | 3 | −0.9 | bad | good | bad | good |
| 68799-57530 | | | | | | |
| 0100876533- | 3 | −1.9 | bad | good | bad | good |
| 68801-57532 | | | | | | |
| 0100876575- | 3 | −1.4 | bad | good | bad | good |
| 68802-57533 | | | | | | |
| 0100876616- | 3 | −1.6 | dubious | good | bad | good |
| 68751-57470 | | | | | | |
| 0100876690- | 3 | −1.7 | dubious | good | bad | good |
| 68743-57462 | | | | | | |
| 0100876773- | 3 | −1.1 | bad | good | bad | good |
| 68803-57534 | | | | | | |
| 0100876814- | 3 | −0.3 | bad | good | bad | good |
| 68805-57536 | | | | | | |
| 0100876856- | 3 | −0.3 | bad | good | bad | good |
| 68804-57535 | | | | | | |
| 0100876898- | 3 | −1.9 | bad | good | bad | good |
| 68807-57538 | | | | | | |
| 0100876939- | 3 | −2.6 | bad | good | bad | good |
| 68807-57538 | | | | | | |
| 0100877052- | 3 | −0.6 | bad | good | bad | good |
| 68744-57463 | | | | | | |
| — | — | bad | 198 (57.9%) | 0 (0%) | 290 (84.8%) | 1 (0.3%) |
| — | — | dubious | 125 (36.5%) | 4 (1.2%) | 15 (4.4%) | 0 (0%) |
| — | — | good | 19 (5.6%) | 338 (98.8%) | 37 (10.8%) | 341 (99.7%) |

### BRIEF DESCRIPTION OF DRAWINGS

FIG. 1: Typical artefacts in mcroarray based hybridisation signals. The plots show the correlation between single or averaged hybridisation profiles. ‘A’ shows a typical chip classified as “good”. The small random deviations from the sample median are due to the approximately normally distributed experimental noise. A typical chip classified as “unacceptable” by visual inspection is shown in ‘B’. Many spots showed no signal, resulting in a log ratio of=after thresholding the signals to X>0. The opposite case is shown in FIG. 1c. This chip has very strong hybridization signals and was classified as “good” by visual inspection However, the hybridization conditions have been too unspecific and most of the oligos were saturated. ‘D’ shows a chip classified as “acceptable”. Hybridization signals were weak compared to background intensity, resulting in a high amount of noise. ‘E’ shows the comparison of group averages over 64 chips in a study hybridised at 42° C. and 48 chips from the same study hybridised at 44° C. ‘F’ shows the comparison of group averages over 447 regular chips from one study and 200 chips with a simulated accidental probe exchange during slide production affecting 12 positions on the chip.

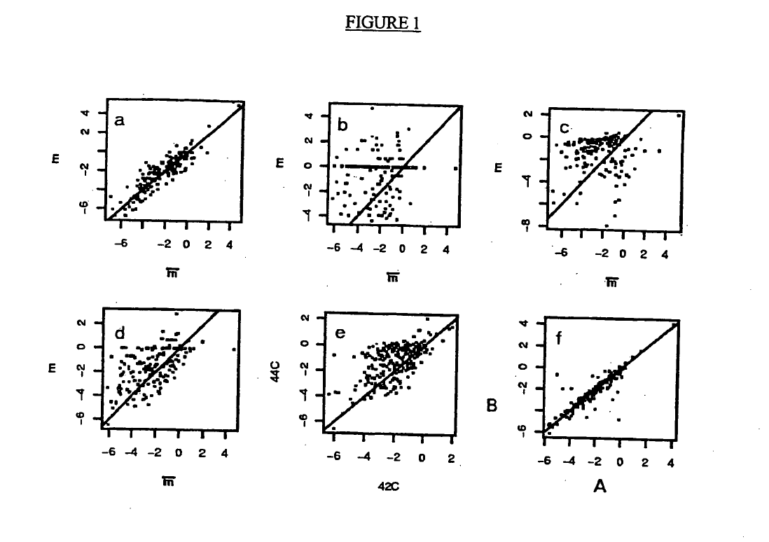

FIG. 2: Comparison between univariate (central rectangle) and mulivariate (ellipse) upper confidence intervals. P₁ is not detected as outlier by univariate tₖ-distance, but by multivariate T²-statistic. P2 is erroneously detected as outlier by the univariate tₖ-distance, but not by multivariate T²-statistic. For P₃ (non-outlier) and P₄ (outlier) both methods give the same decision.

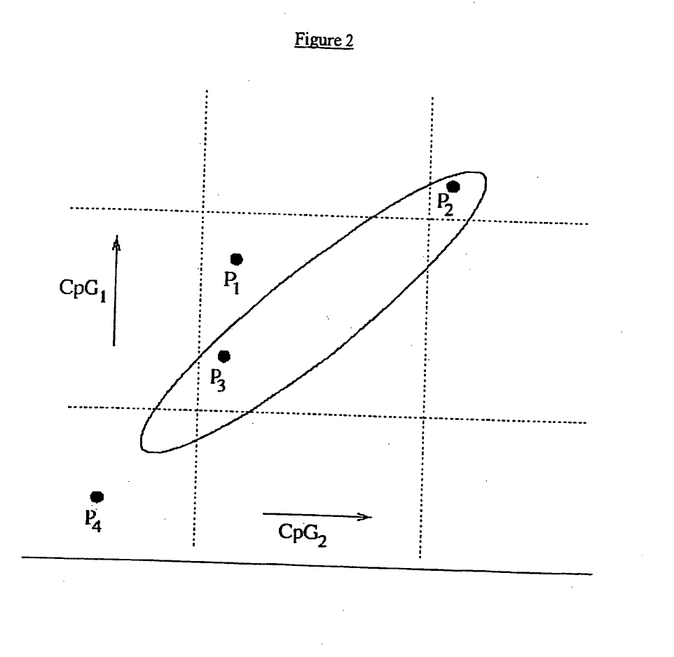

FIG. 3: \tilde{T}²-Distances of robust PCA versus classical PCA for the Lymphoma dataset. The \tilde{T}_{UCL}² values are shown as two dotted lines. Chips to the right of the vertical line were detected as outliers by robust PCA. Chips above the horzontal line were detected as outliers by classical PCA. Chips classified as ‘anacceptable’ by visual inspection are shown as squares, ‘acceptable’ chips as triangles and ‘good’ chips as crosses. Note that ‘goos’ chips detected as outliers by rPCA have all been confirmed to show saturated hybridization signals. The \tilde{T}_{UCL}² values are calculated with d=10 and significance level α=0.025.

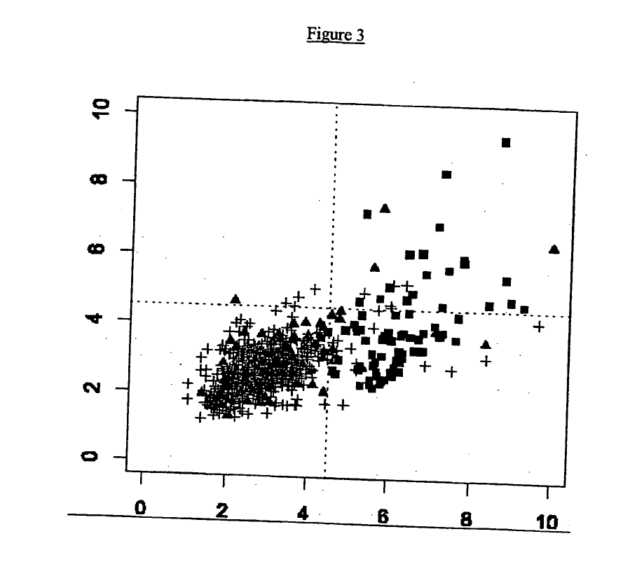

FIG. 4: T² control chart of ALL/AML study. Over the course of the experiment a total of 46 oligomeres for 35 different CpG positions had to be re-synthesized. Oligos were replaced at time indices 234 and 315. The upper plot shows the T-distance of 433 hybridizations, where the grey curve shows the running average as computed by a lowess fit. The lower plot shows the T_{w} and L-distance between HDS and CDS with a window size of N_{HDS}=N_{CDS}=75.

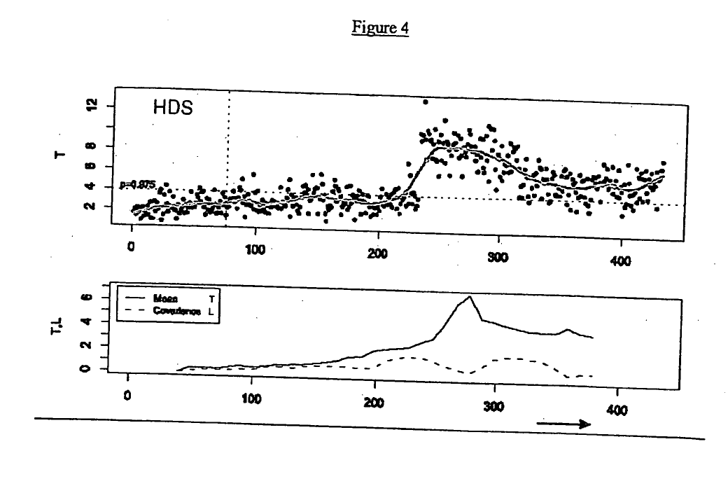

FIG. 5: T² control chart of simulated probe exchange in the Lymphoma data set, Between chips 300 and 500 an accidental oligo probe exchange during slide production was simulaed by rotating 12 randomly selected CpG positions. The upper plot shows the T-distance of all 647 hybridisations, where the line of the curve shows the running averageas computed by a lowess fit. Triangular points are chips classified as ‘unacceptable’ by visual inspection. The lower plot shows the T- and L-distance between HDS and CDS with a window size of N_{HDS}=N_{CDS}=75

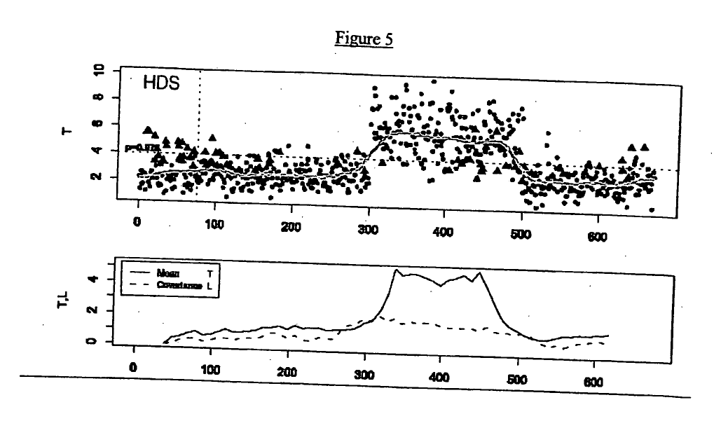

FIG. 6: T² control chart of temperature experiment. The same ALL/AML samples were hybridized at 4 different temperatures. The upper pit shows the T-distance of all 207 hybridizations to the HDS, where the line of the curve shows the running average as computed by a lowess fit. The lower plot shows the T_{w} and L-distance between HDS and CDS with a window size of N_{HDS}=N_{CDS}=30

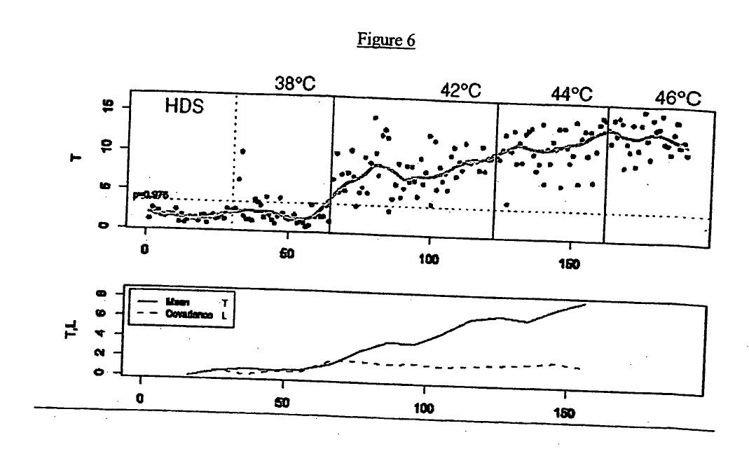

FIG. 7: A panel of box plots, wherein the experimental series described according to Example 2 corresponds to box plot ‘I’. The variable distribution summarized is the 75% quantiles of the standard deviations of the per spot percentage of pixels that surpass the per spot one standard deviation about the mean of all pixel values threshold. The lower horizontal line displays the 75% quantile and the 95% quantile of this distribution calculated from the combined five data sets shown in the individual box plots to the ‘2’ to ‘6’. The thus defined thresholds are used for grading the experimental unit with respect to this single variable.

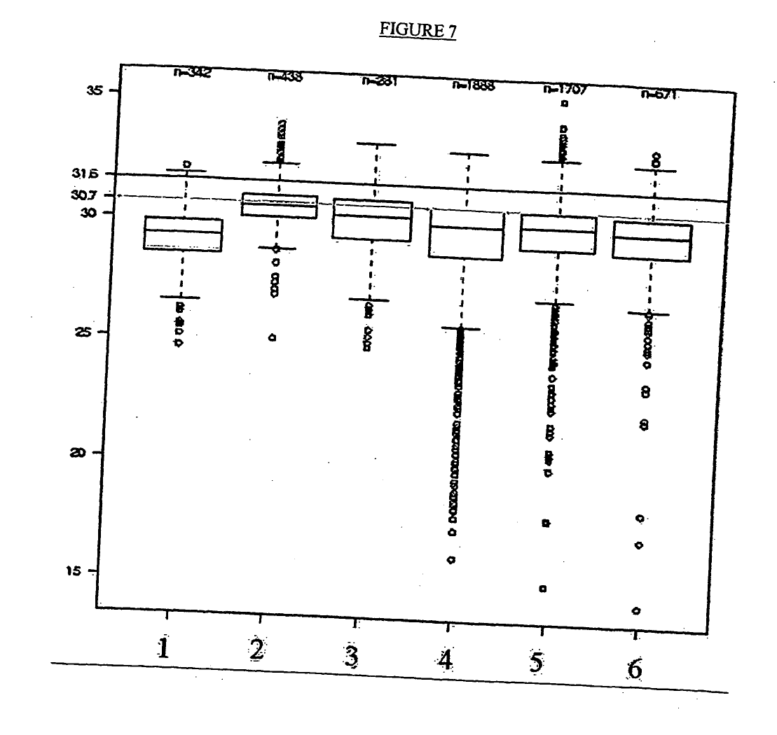

1. A method of verifying and controlling assays for the analysis of nucleic acid variations by means of statistical process control, characterized in that variables of each experiment are monitored by measuring deviations of said variables from a reference data set and wherein said experiments or batches thereof are indicated as unsuitable for further interpretation if they exceed predetermined limits.

2. A method according to claim 1 wherein said nucleic acid variations are cytosine methylation variations.

3. A method according to claim 1 wherein said statistical process control is taken from the group comprising multivariate statistical process control and univariate statistical process control.

4. A method according to claim 1 comprising the following steps:
a) defining a reference data set;
b) defining a test data set;
c) determining the statistical distance between the reference data set and test data set or elements or subsets thereof;
d) identifying individual elements or subsets of the test dataset which have a statistical distance larger than that of a predetermined value.

5. The method according to claim 4, further comprising in step b) reducing the data dimensionality of the reference and test data set by means of robust embedding of the values into a lower dimensional representation.

6. The method according to claim 5 wherein step b) is carried out by calculating the embedding space using one or both of the reference and the test data sets.

7. The method according to claim 4 further comprising,
e) further investigating said identified elements or subsets of the test dataset to determine the contribution of individual variables to the determined statistical distance.

8. The method according to claim 4 further comprising,
e) excluding said identified experiments or batches thereof from further analysis.

9. The method of claim 4 wherein in step d) said statistical distance is calculated by means of one or more methods taken from the group consisting of the Hotelling's T² distance between a single test measurement vector and the reference data set, the Hotelling'-T² distance between a subset of the test data set and the reference data set, the distance between the covariance matrices of a subset of the test data set and the covariance matrix of the reference set, percentiles of the empirical distribution of the reference data set and percentiles of a kernel density estimate of the distribution of the reference data set, distance from the hyperplane of a nu-SVM, estimating the support of the distribution of the reference data set.

10. The method according to claim 5 wherein the data dimensionality reduction is carried out by means of principle component analysis.

11. The method according to claim 5 wherein the data dimensionality reduction step comprises the following steps:
i) Projecting the data set by means of robust principle component analysis;
ii) Removing outliers from the data set according to their statistical distances calculated by means of one or more methods taken from the group consisting of: Hotelling's T² distance; percentiles of the empirical distribution of the reference data set; Percentiles of a kernel density estimate of the distribution of the reference data set and distance from the hyperplane of a nu-SVM, estimating the support of the distribution of the reference data set;
iii) Calculating the embedding projection by standard principle component analysis and projecting the cleared or the complete data set onto this basis vector system.

12. The method according to claim 4 wherein at least one of the variables measured in steps a) and b) is determined according to the methylation state of the nucleic acids.

13. The method according to claim 4 wherein at least one of the variables measured in step a) and b) is determined by the environment used to conduct the assay.

14. The method according to claim 4 to wherein said data sets comprises one or more variables selected from the group comprising mean background/baseline values; scatter of the background/baseline values; scatter of the foreground values, geometrical properties of the array, percentiles of background values of each spot and positive and negative assay control measures.

15. A method according to claim 4 wherein the reference data set is the complete series of experiments being analysed.

16. A method according to claim 4 wherein the reference data set is derived from experiments carried out separately to those of the test data set.

17. A method according to claim 4 wherein the reference data set is derived from a set of experiments wherein the value of each variable of each experiment is either within a predetermined limit or optimally controlled.

18. A method according to claim 4 further comprising the generation of a document comprising said elements or subsets of the test data determined according to step d) of claim 4.

19. A method according to claim 18 wherein said document further comprises the contribution of individual variables to the determined statistical distance.

20. A method according to claim 18 wherein said document is stored on a computer readable format.

21. A method according to one of claims 1 to 20 wherein said method is implemented by means of a computer.

22. A computer program product for the verifying and controlling assays for the analysis of nucleic acid variations comprising:
a) a computer code that receives as input a reference data set;
b) a computer code that receives as input a test data set;
c) a computer code that determines the statistical distance between the reference data set and test data set or elements or subsets thereof;
d) a computer code that identifies individual elements or subsets of the test dataset which have a statistical distance larger than that of a predetermined value; and
e) a computer readable medium that stores the computer code.

23. The computer program product of claim 22 further comprising
f) a computer code that reduces the data dimensionality of the reference and test data set by means of robust embedding of the values into a lower dimensional representation.

24. The computer program product of claim 22 characterised in that the embedding space is calculated using one or both of the reference and the test data sets.

25. The computer program product of claims claim 22 to 24 further comprising,
g) a computer code that investigates said identified elements or subsets of the test dataset to determine the contribution of individual variables to the determined statistical distance.

26. The computer program product of claim 22 wherein said statistical distance is calculated by means of one or more methods taken from the group consisting of the Hotelling's T² distance between a single test measurement vector and the reference data set, the Hotelling'-T² distance between a subset of the test data set and the reference data set, the distance between the covariance matrices of a subset of the test data set and the covariance matrix of the reference set, percentiles of the empirical distribution of the reference data set and percentiles of a kernel density estimate of the distribution of the reference data set, distance from the hyperplane of a nu-SVM, estimating the support of the distribution of the reference data set.

27. The computer program product of claim 23 wherein the data dimensionality reduction is carried out by means of principle component analysis.

28. The computer program product of claim 23, wherein the data dimensionality reduction step comprises the following steps:
(i) Projecting the data set by means of robust principle component analysis;
(ii) Removing outliers from the data set according to their statistical distances calculated by means of one or more methods taken from the group consisting of: Hotelling's T² distance; percentiles of the empirical distribution of the reference data set; Percentiles of a kernel density estimate of the distribution of the reference data set and distance from the hyperplane of a nu-SVM, estimating the support of the distribution of the reference data set;
(iii) Calculating the embedding projection by standard principle component analysis and projecting the cleared or the complete data set onto this basis vector system.

29. The computer program product of claims 22 to 28 further comprising a computer code that generates a document comprising said elements or subsets of the test data which have a statistical distance larger than that of a predetermined value.
